(19) United States
(12) Patent Application Publication
(10) Pub. No.: US 2017/0283791 A1
(43) Pub. Date: Oct. 5, 2017

(54) NUCLEIC ACID-BINDING SOLID-PHASE CARRIER AND NUCLEIC ACID EXTRACTION METHOD

(71) Applicant: Seiko Epson Corporation, Tokyo (JP)

(72) Inventors: Hitoshi FUKUSHIMA, Tsukuba (JP); Yuji SAITO, Shiojiri (JP); Fumio TAKAGI, Chino (JP); Atsushi WATANABE, Hachinohe (JP); Yu MAEDA, Hachinohe (JP)

(73) Assignee: Seiko Epson Corporation, Tokyo (JP)

(21) Appl. No.: 15/472,862

(22) Filed: Mar. 29, 2017

(30) Foreign Application Priority Data
Mar. 30, 2016 (JP) 2016-067702

(51) Int. Cl. C12N 15/10; B03C 1/02

(52) U.S. Cl. CPC C12N 15/1013; B03C 1/02; B03C 2201/18

(57) ABSTRACT

A nucleic acid-binding solid-phase carrier includes a magnetic particle of an amorphous metal containing Fe, Cr, Si, and B, and a silicon oxide film provided on the surface of the magnetic particle.

This application claims the benefit of Japanese Patent Application No. 2016-067702, filed on Mar. 30, 2016. The content of the aforementioned application is incorporated herein by reference in its entirety.

### BACKGROUND

### 1. Technical Field

The present invention relates to a nucleic acid-binding solid-phase carrier and a nucleic acid extraction method.

### 2. Related Art

In recent years, due to the development of technologies utilizing genes, medical treatments utilizing genes such as gene diagnosis or gene therapy have been drawing attention. In addition, many methods using genes in determination of breed varieties or breed improvement have also been developed in agricultural and livestock industries. As technologies for utilizing genes, technologies such as a PCR (Polymerase Chain Reaction) method are widely used. Nowadays, the PCR method has become an indispensable technology for elucidation of information on biological materials.

The PCR method is a method of amplifying a target nucleic acid by subjecting a solution (reaction mixture) containing a nucleic acid to be amplified (target nucleic acid) and a reagent to a thermal cycle. The thermal cycle is a treatment of periodically subjecting the reaction mixture to two or more temperature steps. In the PCR method, a method of performing a two- or three-step thermal cycle is generally performed.

In recent years, as a pretreatment device to be used in the PCR method, a device in which a liquid layer is formed in a cartridge, and a nucleic acid is purified and extracted by allowing a magnetic particle having the nucleic acid bound thereto to pass through the liquid layer has been proposed.

For example, JP-A-9-19292 (Patent Document 1) describes a nucleic acid-binding magnetic carrier which is a magnetic silica particle containing a superparamagnetic metal oxide.

However, in the nucleic acid-binding magnetic carrier described in Patent Document 1, iron oxide is used as the superparamagnetic metal oxide. Due to this, in the nucleic acid-binding magnetic carrier described in Patent Document 1, the responsiveness to an external magnetic field is lacking, and for example, in the case where the direction of application of an external magnetic field is changed at a frequency of several hertz or more, the nucleic acid-binding magnetic carrier sometimes cannot follow the change in the external magnetic field. Therefore, in the case of using such a carrier, for example, in a step of washing the carrier having a nucleic acid adsorbed thereon, the carrier does not follow the change in the external magnetic field, and a nucleic acid sometimes cannot be efficiently extracted.

### SUMMARY

An advantage of some aspects of the invention is to provide a nucleic acid-binding solid-phase carrier capable of efficiently extracting a nucleic acid. Another advantage of some aspects of the invention is to provide a nucleic acid extraction method capable of efficiently extracting a nucleic acid.

A nucleic acid-binding solid-phase carrier according to an aspect of the invention includes a magnetic particle of an amorphous metal containing Fe, Cr, Si, and B, and a silicon oxide film provided on the surface of the magnetic particle.

In such a nucleic acid-binding solid-phase carrier, in the case where the nucleic acid-binding solid-phase carrier having a nucleic acid adsorbed thereon is moved by changing the direction of application of the external magnetic field to effect washing, the followability with respect to the change in the external magnetic field can be improved. Therefore, the nucleic acid-binding solid-phase carrier having a nucleic acid adsorbed thereon can be sufficiently washed by changing the external magnetic field, and thus, the nucleic acid can be efficiently extracted.

In the nucleic acid-binding solid-phase carrier according to the aspect of the invention, it is preferred that the nucleic acid-binding solid-phase carrier has a saturation magnetization of 50 emu/g or more.

Such a nucleic acid-binding solid-phase carrier can follow the change in the external magnetic field.

In the nucleic acid-binding solid-phase carrier according to the aspect of the invention, it is preferred that the nucleic acid-binding solid-phase carrier has a saturation magnetization of 100 emu/g or more and 150 emu/g or less.

Such a nucleic acid-binding solid-phase carrier can more reliably follow the change in the external magnetic field, and also can reduce the variation in the production of the nucleic acid-binding solid-phase carrier.

In the nucleic acid-binding solid-phase carrier according to the aspect of the invention, it is preferred that the magnetic particle has an average particle diameter of 0.5 μm or more and 10 μm or less.

Such a nucleic acid-binding solid-phase carrier can decrease the Ct value in the case where a nucleic acid extracted and purified using the nucleic acid-binding solid-phase carrier is subjected to a gene analysis by a PCR method and also can suppress Brownian motion in the case where washing or the like of a nucleic acid adsorbed on the nucleic acid-binding solid-phase carrier is performed by changing the direction of application of the external magnetic field.

In the nucleic acid-binding solid-phase carrier according to the aspect of the invention, it is preferred that the silicon oxide film has an average thickness of 1 nm or more and 500 nm or less.

Such a nucleic acid-binding solid-phase carrier can increase the saturation magnetization per unit mass and also can reduce the variation in the production of the silicon oxide film.

In the nucleic acid-binding solid-phase carrier according to the aspect of the invention, it is preferred that the magnetic particle is a soft magnetic body.

Such a nucleic acid-binding solid-phase carrier can have a low coercivity.

A nucleic acid extraction method according to an aspect of the invention includes adsorbing a nucleic acid on the nucleic acid-binding solid-phase carrier according to the aspect of the invention in an adsorption liquid containing a chaotropic substance, washing the nucleic acid-binding solid-phase carrier having the nucleic acid adsorbed thereon by placing the nucleic acid-binding solid-phase carrier having the nucleic acid adsorbed thereon in a washing liquid, and eluting the nucleic acid in an eluent by placing the nucleic acid-binding solid-phase carrier having the nucleic acid adsorbed thereon in the eluent, wherein the washing the nucleic acid-binding solid-phase carrier is performed by vibrating the nucleic acid-binding solid-phase carrier having the nucleic acid adsorbed thereon in the washing liquid by a magnetic force.

In such a nucleic acid extraction method, the nucleic acid-binding solid-phase carrier can follow the change in the external magnetic field, and therefore, the nucleic acid-binding solid-phase carrier having a nucleic acid adsorbed thereon can be sufficiently washed by changing the external magnetic field. Accordingly, in such a nucleic acid extraction method, a nucleic acid can be efficiently extracted.

In the nucleic acid extraction method according to the aspect of the invention, it is preferred that the eluting the nucleic acid is performed by vibrating the nucleic acid-binding solid-phase carrier having the nucleic acid adsorbed thereon in the eluent by a magnetic force.

In such a nucleic acid extraction method, the nucleic acid-binding solid-phase carrier can follow the change in the external magnetic field, and therefore, the nucleic acid-binding solid-phase carrier can be directly vibrated by a magnetic force. Accordingly, such a nucleic acid extraction method can improve the elution efficiency (the effect of shaking off the nucleic acid), and can efficiently extract a nucleic acid.

### BRIEF DESCRIPTION OF THE DRAWINGS

The invention will be described with reference to the accompanying drawings, wherein like numbers reference like elements.

FIG. 1 is a cross-sectional view schematically showing a nucleic acid-binding solid-phase carrier according to an embodiment.

FIG. 2 is a flowchart for illustrating a method for producing a nucleic acid-binding solid-phase carrier according to an embodiment.

FIG. 3 is a cross-sectional view schematically showing a cartridge according to an embodiment.

FIG. 4 is a cross-sectional view schematically showing a cartridge according to an embodiment.

FIG. 5 is a flowchart for illustrating a nucleic acid extraction method according to an embodiment.

FIG. 6 is a schematic view for illustrating a nucleic acid extraction method according to an embodiment.

FIG. 7 is a graph showing the magnetization characteristics of a nucleic acid-binding solid-phase carrier of Example 1.

FIG. 8 is a graph showing the magnetization characteristics of a nucleic acid-binding solid-phase carrier of Comparative Example 1.

FIG. 9 is a graph showing the particle size distribution of the nucleic acid-binding solid-phase carrier of Example 1.

FIG. 10 is a graph showing the particle size distribution of the nucleic acid-binding solid-phase carrier of Comparative Example 1.

FIGS. 11A to 11D are schematic views for illustrating an example in which a nucleic acid-binding solid-phase carrier can follow an external magnetic field.

FIGS. 12A to 12D are schematic views for illustrating an example in which a nucleic acid-binding solid-phase carrier cannot follow an external magnetic field.

FIG. 13 is a graph showing a relationship between the average particle diameter of a nucleic acid-binding solid-phase carrier and the Ct value.

### DESCRIPTION OF EXEMPLARY EMBODIMENTS

Hereinafter, preferred embodiments of the invention will be described in detail with reference to the accompanying drawings. Note that the embodiments described below are not intended to unduly limit the content of the invention described in the appended claims. Further, not all the configurations described below are necessarily essential components of the invention.

### 1. Nucleic Acid-Binding Solid-Phase Carrier

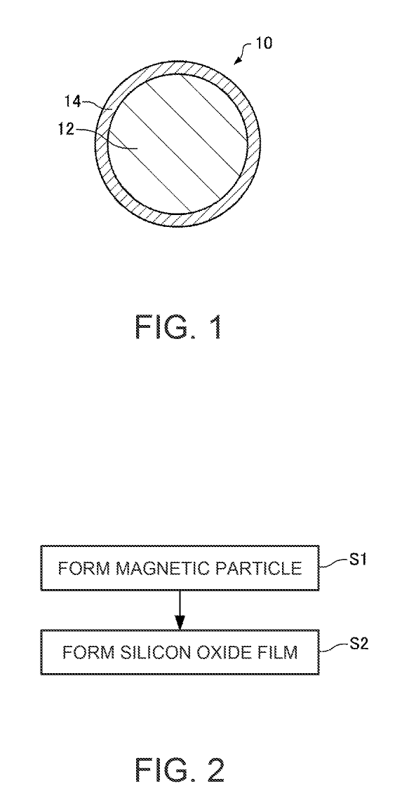

First, the nucleic acid-binding solid-phase carrier according to this embodiment will be described. FIG. 1 is a cross-sectional view schematically showing a nucleic acid-binding solid-phase carrier 10 according to this embodiment.

The nucleic acid-binding solid-phase carrier 10 is a substance capable of holding a nucleic acid such as a DNA (deoxyribonucleic acid) or an RNA (ribonucleic acid) by adsorption, that is, reversible physical binding. In the case where a nucleic acid is extracted, the nucleic acid-binding solid-phase carrier 10 in the form of a powder is used. As shown in FIG. 1, the nucleic acid-binding solid-phase carrier 10 includes a magnetic particle 12 and a silicon oxide film 14.

### 1.1. Magnetic Particle

The magnetic particle 12 is a particle having magnetism. The magnetic particle 12 is an amorphous (noncrystalline) metal. Due to this, the magnetic particle 12 has an irregular atomic arrangement and has almost no crystal structures or crystal grain boundaries therein. Therefore, the magnetic particle 12 hardly causes deformation due to dislocation, destruction starting from a crystal grain boundary, or the like as in the case of a crystalline metal, and has a high hardness. Further, the magnetic particle 12 has an irregular atomic arrangement, and therefore has a relatively high electrical resistance and a low coercivity. Therefore, the magnetic particles 12 are hardly aggregated in a state where a magnetic field is not applied to the magnetic particles 12, and therefore can be uniformly dispersed in a liquid. The magnetic particle 12 is a soft magnetic body. The “soft magnetic body” refers to a body which is relatively easily magnetized or demagnetized among the magnetic bodies, and has a low coercivity and a high magnetic permeability.

The saturation magnetization of the nucleic acid-binding solid-phase carrier 10 is, for example, 50 emu/g or more, and preferably 100 emu/g or more and 150 emu/g or less. The “saturation magnetization” is the value of magnetization which reaches saturation when the applied magnetic field is increased, and in the above description, a value per unit mass is shown. The saturation magnetization of the magnetic particle 12 is, for example, a value which falls within the above range shown as the saturation magnetization of the nucleic acid-binding solid-phase carrier 10.

By setting the saturation magnetization of the nucleic acid-binding solid-phase carrier 10 to 50 emu/g or more, for example, even if the direction of application of the external magnetic field is changed at a frequency of several hertz or more, the nucleic acid-binding solid-phase carrier 10 can follow the change in the external magnetic field. Further, by setting the saturation magnetization of the nucleic acid-binding solid-phase carrier 10 to 150 emu/g or less, the variation in the production of the nucleic acid-binding solid-phase carrier 10 can be reduced. When the saturation magnetization of the nucleic acid-binding solid-phase carrier 10 exceeds 150 emu/g, it becomes difficult to produce the nucleic acid-binding solid-phase carrier 10, and the variation in the production of the nucleic acid-binding solid-phase carrier 10 (for example, the variation in the composition) may sometimes be increased.

The saturation magnetization of the nucleic acid-binding solid-phase carrier 10 or the magnetic particle 12 can be measured using a vibrating sample magnetometer (VSM). Specifically, the measurement can be performed using “TM-VSM 1230-MHHL” manufactured by TAMAKAWA CO., LTD. or the like.

The average particle diameter of the magnetic particle 12 is, for example, 0.5 μm or more and 10 μm or less, and preferably 0.5 μm or more and 5 μm or less. By setting the average particle diameter of the magnetic particle 12 to 0.5 μm or more, the Brownian motion of the nucleic acid-binding solid-phase carrier 10 can be suppressed in a liquid (adsorption liquid). If the nucleic acid-binding solid-phase carrier 10 performs Brownian motion, it becomes difficult for the nucleic acid-binding solid-phase carrier 10 to adsorb a nucleic acid. Further, by setting the average particle diameter of the magnetic particle 12 to 10 μm or less, the Ct (threshold cycle) value at the time of real-time PCR detection can be decreased. The “Ct value” is a cycle number (the number of thermal cycles) at which the amount of an amplification product by PCR has reached a certain predetermined value so that the fluorescent brightness has reached a predetermined value or more. The average particle diameter of the nucleic acid-binding solid-phase carrier 10 is, for example, a value which falls within the above range shown as the average particle diameter of the magnetic particle 12.

The average particle diameter of the nucleic acid-binding solid-phase carrier 10 or the magnetic particle 12 is obtained as a particle diameter (particle diameter D50) when the cumulative frequency from the small diameter side reaches 50% in the particle size distribution on a volume basis obtained by, for example, laser diffractometry.

In an example shown in FIG. 1, the shape of the magnetic particle 12 is a sphere. The shape of the magnetic particle 12 is not particularly limited, and the magnetic particle 12 may have an elliptical or polygonal cross-sectional shape.

The magnetic particle 12 is an amorphous metal (amorphous alloy) containing Fe (iron), Cr (chromium), Si (silicon), and B (boron). The magnetic particle 12 may be constituted by an amorphous metal containing Fe, Cr, Si, and B. In this case, Fe, Cr, Si, and B are contained in a proportion represented by, for example, the following formula (1).

(Fe₁₋ₓCrₓ)ₐ(Si_{1-y}By)₁₀₀₋ₐ (1)

In the formula (1), x and y each represent an atomic ratio, a represents an atomic percentage, and x, y, and a satisfy the following formulae: 0<x≦0.06, 0.3≦y≦0.7, and 70≦a≦81, respectively.

The magnetic particle 12 may further contain C (carbon). The magnetic particle 12 may be constituted by an amorphous metal containing Fe, Cr, Si, B, and C. In this case, Fe, Cr, Si, B, and C are contained in a proportion represented by, for example, the following formula (2).

(Fe₁₋ₓCrₓ)ₐ(Si_{1-y}B_{y})_{100-a-b}C_{b} (2)

In the formula (2), x and y each represent an atomic ratio, a and b each represent an atomic percentage, and x, y, a, and b satisfy the following formulae: 0<x≦0.06, 0.3≦y≦0.7, 70≦a≦81, and 0<b≦2, respectively.

Fe is a main component whose content is the highest in the magnetic particle 12. Therefore, Fe has a great influence on the basic magnetization characteristics and mechanical characteristics of the magnetic particle 12. Fe can, for example, increase the magnetic moment of the magnetic particle 12.

Cr improves the corrosion resistance of the magnetic particle 12. Specifically, by forming a passivation coating composed mainly of an oxide of Cr (such as Cr₂O₃) on the surface of the magnetic particle 12, the corrosion resistance of the magnetic particle 12 is improved. According to this, the oxidation of Fe over time can be suppressed, and therefore, the decrease in the magnetization characteristics of the magnetic particle 12 can be suppressed. By setting x as follows: 0<x≦0.06 as described above, the oxidation of Fe can be suppressed while ensuring the content of Fe.

Si decreases the coercivity of the magnetic particle and increases the magnetic permeability thereof. By setting y and a as follows: 0.3≦y≦0.7 and 70≦a≦81 as described above, respectively, the coercivity of the magnetic particle 12 can be decreased and the magnetic permeability thereof can be increased while ensuring the contents of Fe, Cr, and B.

B decreases the melting point of the amorphous metal. Therefore, the amorphous metal constituting the magnetic particle 12 can be easily produced. Further, B decreases the viscosity of the amorphous metal when melting. Therefore, the magnetic particle 12 can be miniaturized and formed into a spherical shape. In addition, B decreases the coercivity of the magnetic particle 12. By setting y and a as follows: 0.3≦y≦0.7 and 70≦a≦81 as described above, respectively, the melting point of the amorphous metal can be decreased, the magnetic particle 12 can be miniaturized and formed into a spherical shape, and the coercivity of the magnetic particle 12 can be decreased while ensuring the contents of Fe, Cr, and Si.

C decreases the viscosity of the amorphous metal when melting. Therefore, the magnetic particle 12 can be miniaturized and formed into a spherical shape. Further, C decreases the coercivity of the amorphous metal. By setting b as follows: 0<b≦2 as described above, the magnetic particle 12 can be miniaturized and formed into a spherical shape, and the coercivity of the magnetic particle 12 can be decreased while ensuring the contents of Si and B.

The compositional ratio of the magnetic particle 12 can be determined by, for example, ICP optical emission spectrometry specified in JIS G 1258 or spark optical emission spectrometry specified in JIS G 1253. Specifically, for example, an optical emission spectrometer for solids (spark optical emission spectrometer, model: SPECTROLAB, type: LAVMB08A) manufactured by SPECTRO Analytical Instruments GmbH or an ICP device (model: CIROS-120) manufactured by Rigaku Corporation can be used. When C is determined, particularly, an infrared absorption method after combustion in a current of oxygen (after combustion in a high-frequency induction heating furnace) specified in JIS G 1211 is also used. Specifically, a carbon-sulfur analyzer, CS-200 manufactured by LECO Corporation can be used.

In addition, by using X-ray diffractometry, it is possible to determine whether or not the constituent material of the magnetic particle is amorphous. In general, in the case where a clear diffraction peak is not observed, the constituent material can be determined to be amorphous.

The magnetic particle 12 may contain an inevitable element. The inevitable element is an element (impurity) which is unintentionally mixed in the raw material of the magnetic particle 12 or when producing the nucleic acid-binding solid-phase carrier 10. The inevitable element is not particularly limited, however, examples thereof include N (nitrogen), P (phosphorus), S (sulfur), Na (sodium), Mg (magnesium), Al (aluminum), K (potassium), Ca (calcium), Ti (titanium), V (vanadium), Co (cobalt), Ni (nickel), Cu (copper), Zn (zinc), Ga (gallium), Ge (germanium), As (arsenic), Se (selenium), Y (yttrium), Zr (zirconium), Nb (niobium), Mo (molybdenum), Ag (silver), Cd (cadmium), In (indium), Sn (tin), Sb (antimony), and Te (tellurium). In the case where an inevitable element is mixed in the magnetic particle 12, the mixing amount thereof is preferably less than 0.2 mass %, more preferably 0.1 mass % or less of the amount of the amorphous metal constituting the magnetic particle 12.

### 1.2. Silicon Oxide Film

The silicon oxide film 14 specifically adsorbs a nucleic acid in an aqueous solution containing a chaotropic substance. The silicon oxide film 14 is provided on the surface of the magnetic particle 12 as shown in FIG. 1. The silicon oxide film 14 coats the surface of the magnetic particle 12. The material of the silicon oxide film 14 is, for example, SiOₓ (0<x≦2), and is specifically SiO₂. In the example shown in the drawing, the silicon oxide film 14 coats the entire surface of the magnetic particle 12, however, the silicon oxide film 14 may be provided on at least part of the surface of the magnetic particle 12.

The average thickness of the silicon oxide film 14 is, for example, 1 nm or more and 500 nm or less, and preferably 10 nm or more and 300 nm or less. The “average thickness” is the average of the thicknesses of the silicon oxide films 14 of a plurality of nucleic acid-binding solid-phase carriers 10 in the case where a nucleic acid is extracted using the nucleic acid-binding solid-phase carrier 10 in the form of a powder. In the example shown in the drawing, the silicon oxide film 14 coats the surface of the magnetic particle 12 with a uniform thickness, however, the silicon oxide film 14 may have a different thickness on the surface of the magnetic particle 12. In this case, the “average thickness” is the average of the maximum thickness of the silicon oxide film 14 in each nucleic acid-binding solid-phase carrier 10.

By setting the average thickness of the silicon oxide film 14 to 1 nm or more, the variation in the production of the silicon oxide film 14 can be reduced. When the average thickness of the silicon oxide film 14 is less than 1 nm, it becomes difficult to produce the silicon oxide film 14, and the variation in the production of the silicon oxide film 14 (for example, the variation in the thickness) may sometimes be increased. Further, by setting the average thickness of the silicon oxide film 14 to 500 nm or less, the saturation magnetization per unit mass of the nucleic acid-binding solid-phase carrier 10 can be increased.

The thickness of the silicon oxide film 14 can be determined by, for example, measuring the thickness in a non-contact manner using light such as infrared light, or by measuring the thickness of the silicon oxide film 14 from a cross-sectional SEM (scanning electron microscope) image of the nucleic acid-binding solid-phase carrier 10.

The silicon oxide film 14 may contain an inevitable element. Examples of the inevitable element contained in the silicon oxide film 14 include the same elements as those listed above as the inevitable element contained in the magnetic particle 12.

### 2. Method for Producing Nucleic Acid-Binding Solid-Phase Carrier

Next, a method for producing the nucleic acid-binding solid-phase carrier 10 according to this embodiment will be described with reference to the accompanying drawings. FIG. 2 is a flowchart for illustrating a method for producing the nucleic acid-binding solid-phase carrier 10 according to this embodiment.

First, a magnetic particle 12 is formed (Step S1). The magnetic particle 12 is formed by, for example, any of a variety of powdering methods such as atomization methods (such as a water atomization method, a gas atomization method, and a spinning water atomization method) and pulverization methods, or the like.

The atomization methods are divided into a water atomization method, a gas atomization method, and a spinning water atomization method according to the difference in the type of a cooling medium or the configuration of a device. The atomization method is a method in which a molten metal (metal melt) is caused to collide with a fluid (liquid or gas) jetted at a high speed to atomize the metal melt into a fine powder and also to cool the fine powder, whereby a metal powder (amorphous alloy powder) is produced. By producing the amorphous alloy powder through such an atomization method, an extremely fine powder can be efficiently produced. Further, the shape of the particle of the amorphous alloy powder is closer to a spherical shape by the action of surface tension.

Further, in the spinning water atomization method, the metal melt can be cooled at an extremely high speed, and therefore can be solidified in a state where random atomic arrangement in the molten metal is highly maintained. Due to this, by the spinning water atomization method, an amorphous alloy powder having a particularly high amorphization degree (an alloy powder which is more reliably amorphized) can be efficiently produced. Therefore, as the method of forming the magnetic particle 12, the spinning water atomization method is preferred.

Specifically, in the spinning water atomization method, a cooling liquid is supplied by ejection along the inner circumferential surface of a cooling cylindrical body, and is spun along the inner circumferential surface of the cooling cylindrical body, whereby a cooling liquid layer is formed on the inner circumferential surface. On the other hand, the raw material of the amorphous alloy is melted, and while allowing the obtained molten metal to freely fall, a liquid or gas jet is blown to the molten metal. By doing this, the molten metal is scattered, and the scattered molten metal is incorporated into the cooling liquid layer. As a result, the molten metal which is atomized by scattering is solidified by rapid cooling, and therefore, the amorphous alloy powder is obtained.

Subsequently, a silicon oxide film 14 is formed on the surface of the magnetic particle 12 (Step S2). The silicon oxide film 14 is formed by, for example, a sol-gel method, a CVD (Chemical Vapor Deposition) method, a sputtering method, a laser ablation method, or the like.

By the steps described above, the nucleic acid-binding solid-phase carrier 10 can be produced.

### 2. Cartridge

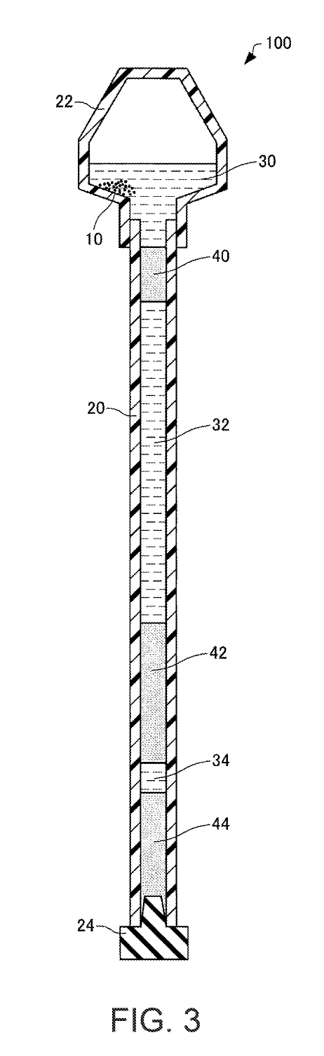

Next, a cartridge according to this embodiment will be described. FIG. 3 is a cross-sectional view schematically showing a cartridge 100 according to this embodiment.

The cartridge 100 is a container for adsorbing a nucleic acid on the nucleic acid-binding solid-phase carrier (for example, the nucleic acid-binding solid-phase carrier 10) according to the invention, and purifying the nucleic acid by washing the nucleic acid-binding solid-phase carrier having the nucleic acid adsorbed thereon, and separating and eluting the nucleic acid from the nucleic acid-binding solid-phase carrier having the nucleic acid adsorbed thereon.

As shown in FIG. 3, the cartridge 100 includes, for example, a first tube 20, a second tube 22, and a plug 24.

The shape of the first tube 20 is, for example, a cylindrical shape. The inner diameter of the first tube 20 is, for example, 1 mm or more and 5 mm or less. In one end portion of the first tube 20, the second tube 22 is provided. The shape of the second tube 22 is, for example, a vessel-like shape. In the other end portion of the first tube 20, the plug 24 is provided. The second tube 22 and the plug 24 are connected to the first tube 20 so as to hermetically seal the space in the first tube 20 and the space in the second tube 22. The material of the tubes 20 and 22 is, for example, a resin such as polypropylene. The material of the plug 24 is, for example, rubber.

As shown in FIG. 3, the cartridge 100 holds an adsorption liquid 30, a washing liquid 32, an eluent 34, a first oil 40, a second oil 42, and a third oil 44. In the example shown in the drawing, the adsorption liquid 30, the first oil 40, the washing liquid 32, the second oil 42, the eluent 34, and the third oil 44 are placed in the cartridge 100 in this order from the second tube 22 side to the plug 24 side. Specifically, the adsorption liquid 30 is placed in the second tube 22, and the washing liquid 32, the eluent 34, and the oils 40, 42, and 44 are placed in the first tube 20.

The adsorption liquid 30 is a liquid to serve as a place where a nucleic acid is adsorbed on the nucleic acid-binding solid-phase carrier 10. The adsorption liquid 30 is an aqueous solution containing a chaotropic substance. As the adsorption liquid 30, for example, a liquid containing 5 M guanidine thiocyanate, 2% Triton X-100, and 50 mM Tris-HCl (pH 7.2) is used.

The chaotropic substance is a substance which generates a chaotropic ion (a monovalent anion having a large ionic radius) in an aqueous solution, has an activity to increase the water solubility of a hydrophobic molecule, and contributes to the adsorption of a nucleic acid on the nucleic acid-binding solid-phase carrier 10. Specifically, as the chaotropic substance, guanidine hydrochloride, sodium iodide, sodium perchlorate, or the like is used.

By the existence of the chaotropic substance in the adsorption liquid 30, a nucleic acid in the adsorption liquid 30 is more thermodynamically advantageous when it exists in a state of being adsorbed on a solid than when it exists in a state of being surrounded by an aqueous solution. Therefore, a nucleic acid is adsorbed on the surface of the nucleic acid-binding solid-phase carrier 10.

The washing liquid 32 is a liquid for washing the nucleic acid-binding solid-phase carrier 10 having a nucleic acid adsorbed thereon. The washing liquid 32 is, for example, a low-salt concentration aqueous solution such as a buffer. The salt concentration in the low-salt concentration aqueous solution is, for example, 100 mM or less. The salt to be used for preparing the washing liquid 32 as a buffer solution is, for example, Tris, HEPES, PIPES, phosphoric acid, or the like. Further, the washing liquid 32 may contain an alcohol.

Although not shown in the drawing, the washing liquid may be separated into a plurality of portions. Specifically, the washing liquid may be separated into a first portion, a second portion, and a third portion by an oil. In this case, the first portion, the second portion, and the third portion may have different compositions or concentrations. According to this, the nucleic acid-binding solid-phase carrier 10 having a nucleic acid adsorbed thereon can be more reliably washed.

The eluent 34 is a liquid for separating a nucleic acid from the nucleic acid-binding solid-phase carrier 10 having the nucleic acid adsorbed thereon and eluting the nucleic acid in the eluent 34. As the eluent 34, for example, pure water is used. The eluent 34 may be a liquid droplet.

The first oil 40 is placed between the adsorption liquid 30 and the washing liquid 32. The first oil 40 is a liquid which causes phase separation of the adsorption liquid 30 and the washing liquid 32 from each other and is immiscible with the adsorption liquid 30 and the washing liquid 32. The second oil 42 is placed between the washing liquid 32 and the eluent 34. The second oil 42 is a liquid which causes phase separation of the washing liquid 32 and the eluent 34 from each other and is immiscible with the washing liquid 32 and the eluent 34. The third oil 44 is placed between the eluent 34 and the plug 24. The third oil 44 is a liquid which is immiscible with the eluent 34.

As the first oil 40, the second oil 42, and the third oil 44, for example, a silicone-based oil such as dimethyl silicone oil, a paraffin-based oil, a mineral oil, or a mixture thereof is used.

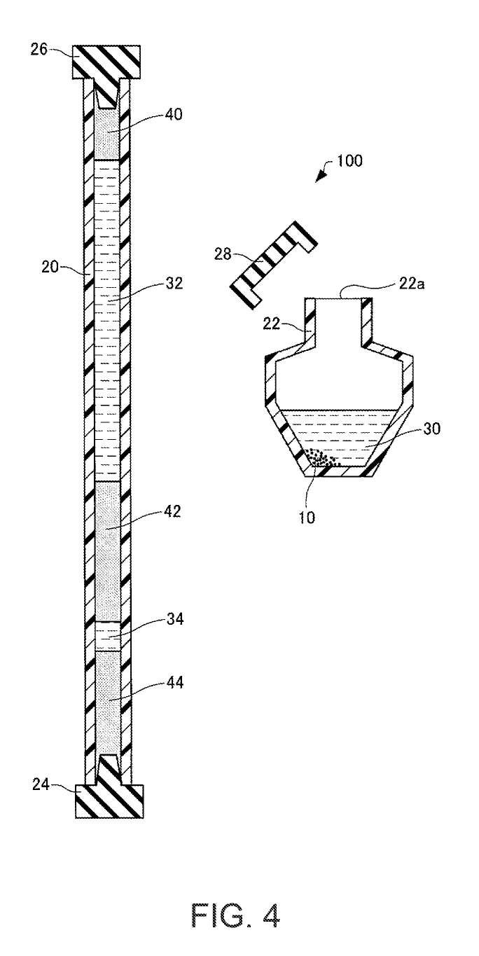

As shown in FIG. 4, in the cartridge 100, the first tube 20 and the second tube 22 are separable from each other. A plug 26 is attachable to and detachable from one end portion of the first tube 20. The second tube 22 has an opening 22a. A plug 28 is attachable to and detachable from the opening 22a.

In the case where the cartridge 100 is assembled, the plug 28 is detached from the second tube 22, and a nucleic acid is introduced into the adsorption liquid 30 having the nucleic acid-binding solid-phase carrier 10 placed therein. Then, the plug 26 is detached from the first tube 20, and one end portion of the first tube 20 is inserted into the opening 22a of the second tube 22. In this manner, the cartridge 100 shown in FIG. 3 can be assembled.

### 3. Nucleic Acid Extraction Method

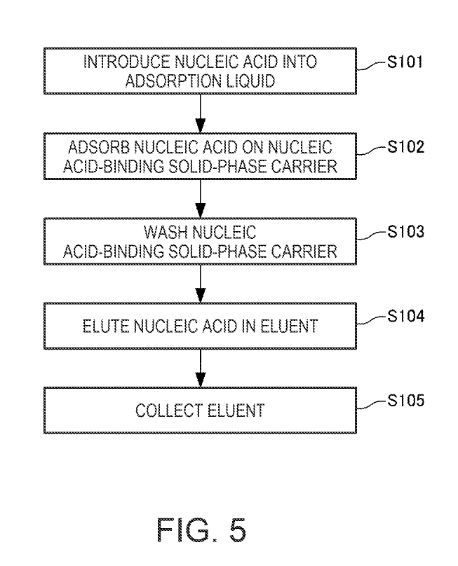

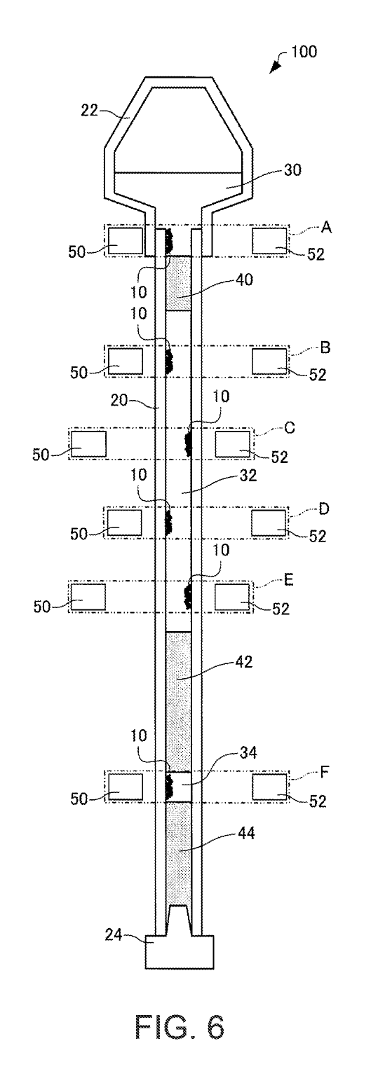

Next, a nucleic acid extraction method according to this embodiment will be described. FIG. 5 is a flowchart for illustrating the nucleic acid extraction method according to this embodiment. FIG. 6 is a schematic view for illustrating the nucleic acid extraction method according to this embodiment. Hereinafter, as one example, a nucleic acid extraction method using the cartridge 100 will be described.

(1) First, as shown in FIG. 4, the plug 28 is detached from the second tube 22, and a nucleic acid is introduced into the adsorption liquid 30 held in the second tube 22 (Step S101). In the adsorption liquid 30, the nucleic acid-binding solid-phase carrier 10 is placed. The nucleic acid is collected from a cell derived from a living organism such as a human or a bacterium, a virus, or the like using a collecting tool such as a cotton swab. After the nucleic acid is introduced into the adsorption liquid 30, the opening 22a of the second tube 22 is closed by the plug 28.

(2) Next, the nucleic acid is placed in the adsorption liquid 30 for the nucleic acid-binding solid-phase carrier 10, and the nucleic acid is adsorbed on the nucleic acid-binding solid-phase carrier 10 in the adsorption liquid 30 (Step S102). The nucleic acid is adsorbed on the surface of the nucleic acid-binding solid-phase carrier 10 by the action of the chaotropic substance contained in the adsorption liquid 30. The nucleic acid is adsorbed on the nucleic acid-binding solid-phase carrier 10 by, for example, stirring the nucleic acid-binding solid-phase carrier 10.

(3) Subsequently, the nucleic acid-binding solid-phase carrier 10 having the nucleic acid adsorbed thereon is placed in the washing liquid 32 so as to wash the nucleic acid-binding solid-phase carrier 10 having the nucleic acid adsorbed thereon (Step S103). Specifically, as shown in FIG. 6, a pair of magnets 50 and 52 are used, and the nucleic acid-binding solid-phase carrier 10 having the nucleic acid adsorbed thereon is moved from the inside of the adsorption liquid 30 to the inside of the washing liquid 32 by the magnetic force of the magnets 50 and 52. The magnets 50 and 52 are, for example, permanent magnets. The pair of magnets 50 and 52 are provided so as to interpose the cartridge 100 between the magnets 50 and 52. As shown in FIG. 6, by bringing the magnet 50 closer to the cartridge 100, the nucleic acid-binding solid-phase carrier 10 is attracted to the inner wall of the cartridge 100 on the magnet 50 side (a state A in FIG. 6). By moving the pair of magnets 50 and 52 to the plug 24 side while keeping this state, the nucleic acid-binding solid-phase carrier 10 can be moved from the inside of the adsorption liquid 30 to the inside of the washing liquid 32.

The step of washing the nucleic acid-binding solid-phase carrier 10 (Step S103) is performed by vibrating the nucleic acid-binding solid-phase carrier 10 having the nucleic acid adsorbed thereon in the washing liquid 32 by the magnetic force. Specifically, while keeping the distance between the magnets 50 and 52 constant, the magnets 50 and 52 are vibrated at a predetermined frequency in the short-side direction orthogonal to the long-side direction (a direction from one end portion to the other end portion of the first tube 20) of the cartridge 100 (hereinafter also simply referred to as “short-side direction”). The magnets 50 and 52 are moved in the long-side direction of the cartridge 100 (hereinafter also simply referred to as “long-side direction”) while keeping this state. That is, the magnets 50 and 52 are moved in the long-side direction while repeating a step of bringing the magnet 50 closer to the cartridge 100 than the magnet 52 and a step of bringing the magnet 52 closer to the cartridge 100 than the magnet 50 (while changing the direction of application of the external magnetic field to the nucleic acid-binding solid-phase carrier 10 at a predetermined frequency).

The magnetic particle 12 of the nucleic acid-binding solid-phase carrier 10 has magnetism as described above. Therefore, when the magnet 50 comes close to the cartridge 100, the nucleic acid-binding solid-phase carrier 10 is attracted to the inner wall (of the first tube 20) of the cartridge 100 on the magnet 50 side (states B and D in FIG. 6). On the other hand, when the magnet 52 comes close to the cartridge 100, the nucleic acid-binding solid-phase carrier 10 is attracted to the inner wall of the cartridge 100 on the magnet 52 side (states C and E in FIG. 6). In the example shown in the drawing, the nucleic acid-binding solid-phase carrier 10 moves in the washing liquid 32 in the following order: B→C→D→E. In this manner, the nucleic acid-binding solid-phase carrier 10 moves in the long-side direction while vibrating in the short-side direction accompanying the vibration in the short-side direction and the movement in the long-side direction of the magnets 50 and 52.

The frequency of vibration of the magnets 50 and 52 in the short-side direction is, for example, 3 Hz or more and 30 Hz or less, and preferably 5 Hz or more and 15 Hz or less. By setting the frequency of vibration of the magnets 50 and 52 to 3 Hz or more, the effect of washing with the washing liquid 32 can be increased. Further, by setting the frequency of vibration of the magnets 50 and 52 to 30 Hz or less, the nucleic acid-binding solid-phase carrier 10 can be made to follow the vibration of the magnets 50 and 52. The moving speed of the magnets 50 and 52 in the long-side direction is not particularly limited as long as the nucleic acid-binding solid-phase carrier 10 can be washed. The vibration in the short-side direction and the movement in the long-side direction of the magnets 50 and 52 are automatically performed by, for example, a moving mechanism (not shown).

(4) Subsequently, the nucleic acid-binding solid-phase carrier 10 having the nucleic acid adsorbed thereon is placed in the eluent 34, and the nucleic acid is eluted in the eluent 34 (Step S104). Specifically, by moving the magnets 50 and 52 in the long-side direction, the nucleic acid-binding solid-phase carrier 10 having the nucleic acid adsorbed thereon is moved from the inside of the washing liquid 32 to the inside of the eluent 34. When the nucleic acid-binding solid-phase carrier 10 having the nucleic acid adsorbed thereon comes in contact with the eluent 34, the nucleic acid is separated from the nucleic acid-binding solid-phase carrier 10 and eluted in the eluent 34.

The step of eluting the nucleic acid (Step S104) is performed by, for example, vibrating the nucleic acid-binding solid-phase carrier 10 having the nucleic acid adsorbed thereon in the eluent 34 by the magnetic force in the same manner as in the step of washing the nucleic acid-binding solid-phase carrier 10 (Step S103). In this step, the magnets 50 and 52 may be vibrated in the short-side direction in a state where the movement of the magnets 50 and 52 in the long-side direction is stopped.

Incidentally, in the step of adsorbing the nucleic acid (Step S102), the nucleic acid-binding solid-phase carrier 10 may be vibrated in the adsorption liquid 30 by the magnetic force in the same manner as in the step of washing the nucleic acid-binding solid-phase carrier 10 (Step S103) when the nucleic acid is adsorbed on the nucleic acid-binding solid-phase carrier 10 by stirring the nucleic acid-binding solid-phase carrier 10. According to this, the adsorption efficiency of the nucleic acid on the nucleic acid-binding solid-phase carrier 10 can be improved.

(5) Subsequently, the plug 24 is detached from the first tube 20, and the eluent 34 in which the nucleic acid has been eluted is collected by, for example, a known method (Step S105).

In this manner, the purified nucleic acid can be extracted.

The extracted nucleic acid is introduced into a container (not shown), and PCR is performed. By doing this, the nucleic acid can be amplified.

### 4. Features

The nucleic acid-binding solid-phase carrier 10 has, for example, the following features.

The nucleic acid-binding solid-phase carrier 10 includes the magnetic particle 12 of an amorphous metal containing Fe, Cr, Si, and B, and the silicon oxide film 14 provided on the surface of the magnetic particle 12. Therefore, the magnetic particle 12 has a high saturation magnetization and a low coercivity. Due to this, in the case where the nucleic acid-binding solid-phase carrier 10 having a nucleic acid adsorbed thereon is moved by changing the direction of application of the external magnetic field to effect washing, the followability of the nucleic acid-binding solid-phase carrier 10 with respect to the change in the external magnetic field can be improved. Therefore, the nucleic acid-binding solid-phase carrier 10 having a nucleic acid adsorbed thereon can be sufficiently washed by changing the external magnetic field, and thus, the nucleic acid can be efficiently extracted. As a result, the extraction amount of the nucleic acid can be increased, and therefore, the speed and sensitivity of PCR can be increased. In addition, the nucleic acid can be extracted in a short time.

For example, if the nucleic acid-binding solid-phase carrier does not follow the change in the external magnetic field, the nucleic acid-binding solid-phase carrier is aggregated to form a block, and therefore, the ratio of a portion which does not come in contact with the washing liquid on the surface of the nucleic acid-binding solid-phase carrier is increased. Due to this, the nucleic acid-binding solid-phase carrier cannot be sufficiently washed in some cases.

The nucleic acid-binding solid-phase carrier 10 has a saturation magnetization of 50 emu/g or more. Therefore, the nucleic acid-binding solid-phase carrier 10 can follow the change in the external magnetic field.

The nucleic acid-binding solid-phase carrier 10 has a saturation magnetization of 100 emu/g or more and 150 emu/g or less. Therefore, the nucleic acid-binding solid-phase carrier 10 can more reliably follow the change in the external magnetic field, and also can reduce the variation in the production of the nucleic acid-binding solid-phase carrier 10.

In the nucleic acid-binding solid-phase carrier 10, the magnetic particle 12 has an average particle diameter of 0.5 μm or more and 10 μm or less. Therefore, the nucleic acid-binding solid-phase carrier 10 can decrease the Ct value in the case where a nucleic acid extracted and purified using the nucleic acid-binding solid-phase carrier 10 is subjected to a gene analysis by a PCR method and also can suppress Brownian motion in the case where a nucleic acid adsorbed on the nucleic acid-binding solid-phase carrier 10 is washed or the like by changing the direction of application of the external magnetic field.

In the nucleic acid-binding solid-phase carrier 10, the silicon oxide film 14 has an average thickness of 1 nm or more and 500 nm or less. Therefore, the nucleic acid-binding solid-phase carrier 10 can increase the saturation magnetization per unit mass of the nucleic acid-binding solid-phase carrier 10 and also can reduce the variation in the production of the silicon oxide film 14.

In the nucleic acid-binding solid-phase carrier 10, the magnetic particle 12 is a soft magnetic body. Therefore, the magnetic particle 12 can have a low coercivity.

The nucleic acid extraction method according to this embodiment has, for example, the following features.

In the nucleic acid extraction method according to this embodiment, a step of washing the nucleic acid-binding solid-phase carrier 10 (Step S103) is performed by vibrating the nucleic acid-binding solid-phase carrier 10 having a nucleic acid adsorbed thereon in the washing liquid 32 by a magnetic force. The nucleic acid-binding solid-phase carrier 10 can follow the change in the external magnetic field (the change in the direction of application of the external magnetic force), and therefore, in the nucleic acid extraction method according to this embodiment, the nucleic acid-binding solid-phase carrier 10 having a nucleic acid adsorbed thereon can be sufficiently washed by changing the external magnetic field. Accordingly, in the nucleic acid extraction method according to this embodiment, a nucleic acid can be efficiently extracted.

In the nucleic acid extraction method according to this embodiment, a step of eluting a nucleic acid (Step S104) is performed by vibrating the nucleic acid-binding solid-phase carrier 10 having the nucleic acid adsorbed thereon in the eluent 34 by a magnetic force. The nucleic acid-binding solid-phase carrier 10 can follow the change in the external magnetic field, and therefore, in the nucleic acid extraction method according to this embodiment, the nucleic acid-binding solid-phase carrier 10 can be directly vibrated by a magnetic force. Therefore, the nucleic acid extraction method according to this embodiment can increase the shear stress of the nucleic acid-binding solid-phase carrier 10 to the eluent as compared with the case where a nucleic acid is eluted by vibrating the entire cartridge using, for example, a vortex mixer, and thus, can improve the elution efficiency (the effect of shaking off the nucleic acid). As a result, in the nucleic acid extraction method according to this embodiment, a nucleic acid can be efficiently extracted.

### 5. Experimental Example

Hereinafter, the invention will be more specifically described by showing experimental examples below. However, the invention is by no means limited to the following experimental examples.

### 5.1. Preparation of Sample

In Example 1, as a magnetic particle, an amorphous alloy powder composed of (Fe_{0.97}Cr_{0.03})₇₆(Si_{0.5}B_{0.5})₂₂C₂ was produced by a spinning water atomization method. On the surface of the magnetic particle, a silicon oxide (SiO₂) film (having an average thickness of about 100 nm) was formed by a sol-gel method, whereby a nucleic acid-binding solid-phase carrier (bead) of Example 1 was obtained.

In Comparative Example 1, a nucleic acid-binding solid-phase carrier was obtained in the same manner as in Example 1 except that a metal powder (manufactured by TOYOBO CO., LTD.) composed of iron oxide was used as the magnetic particle.

In Comparative Example 2, a nucleic acid-binding solid-phase carrier was obtained in the same manner as in Example 1 except that a metal powder (manufactured by chemicell GmbH) composed of iron oxide was used as the magnetic particle.

### 5.2. Evaluation of Magnetization Characteristics

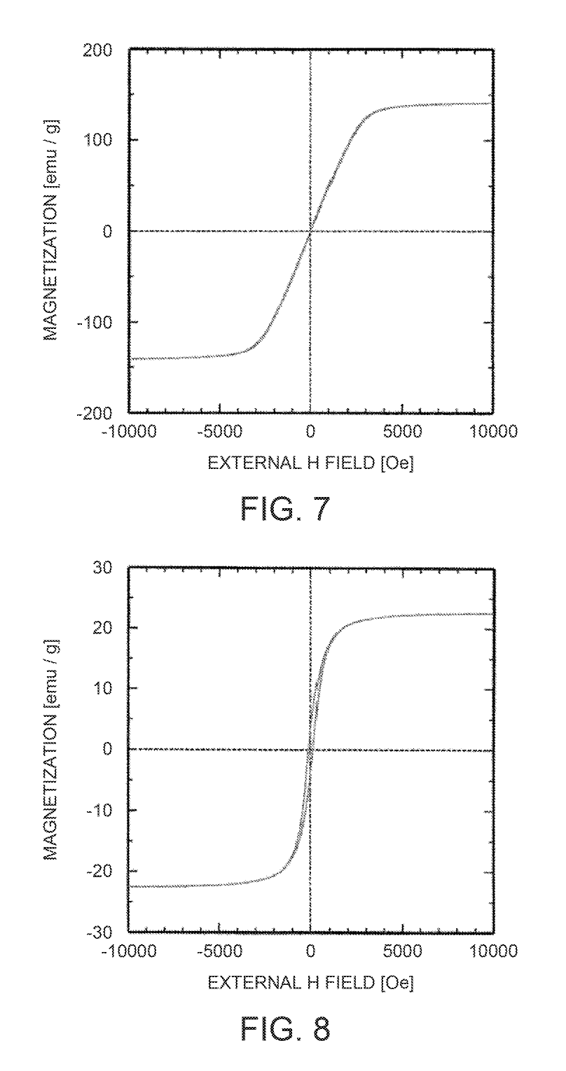

The magnetization characteristics of the nucleic acid-binding solid-phase carriers of Example 1 and Comparative Example 1 were evaluated using a VSM. FIG. 7 is a graph showing the magnetization characteristics of the nucleic acid-binding solid-phase carrier of Example 1. FIG. 8 is a graph showing the magnetization characteristics of the nucleic acid-binding solid-phase carrier of Comparative Example 1.

As shown in FIGS. 7 and 8, the saturation magnetization of the nucleic acid-binding solid-phase carrier of Example 1 was about 140 emu/g, and the saturation magnetization of the nucleic acid-binding solid-phase carrier of Comparative Example 1 was about 22 emu/g. Therefore, it was found that the amorphous alloy powder containing Fe, Cr, Si, and B has a higher saturation magnetization than the metal powder composed of iron oxide.

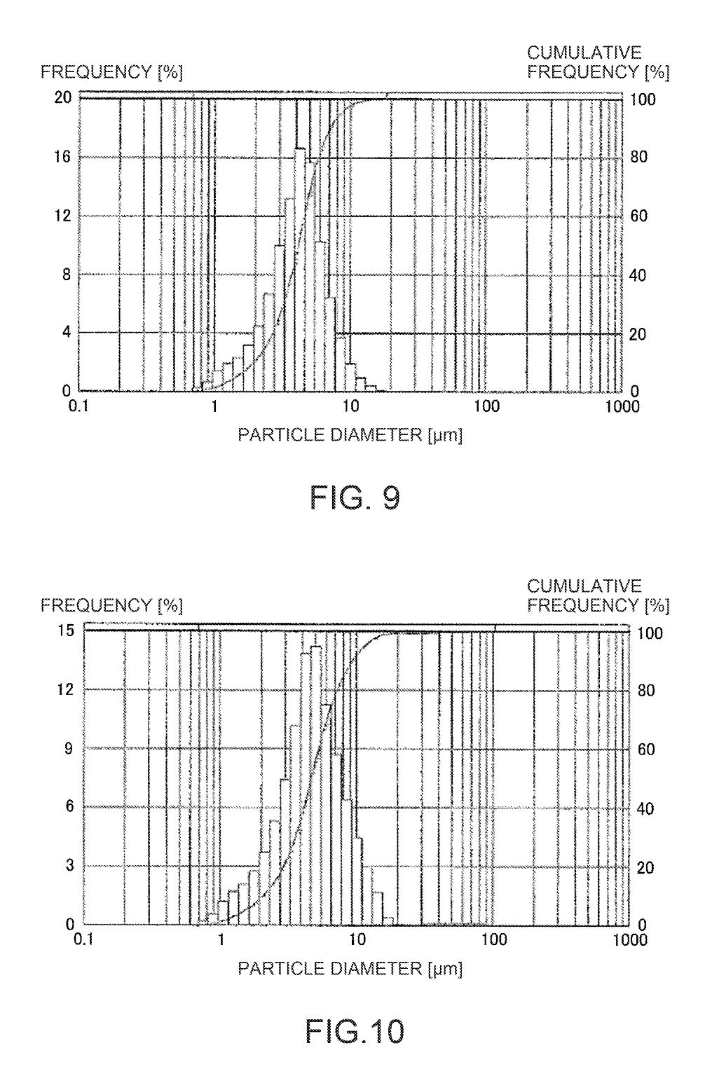

FIG. 9 is a graph showing the particle size distribution of the nucleic acid-binding solid-phase carrier of Example 1 evaluated in FIG. 7. FIG. 10 is a graph showing the particle size distribution of the nucleic acid-binding solid-phase carrier of Comparative Example 1 evaluated in FIG. 8. The average particle diameter of the nucleic acid-binding solid-phase carrier of Example 1 shown in FIG. 9 was 3.4 μm. The average particle diameter of the nucleic acid-binding solid-phase carrier of Comparative Example 1 shown in FIG. 10 was 3.8 μm. The average particle diameter is a particle diameter when the cumulative frequency from the small diameter side reaches 50% in the particle size distribution on a volume basis obtained by laser diffractometry.

5.3. Evaluation of Followability with Respect to External Magnetic Field

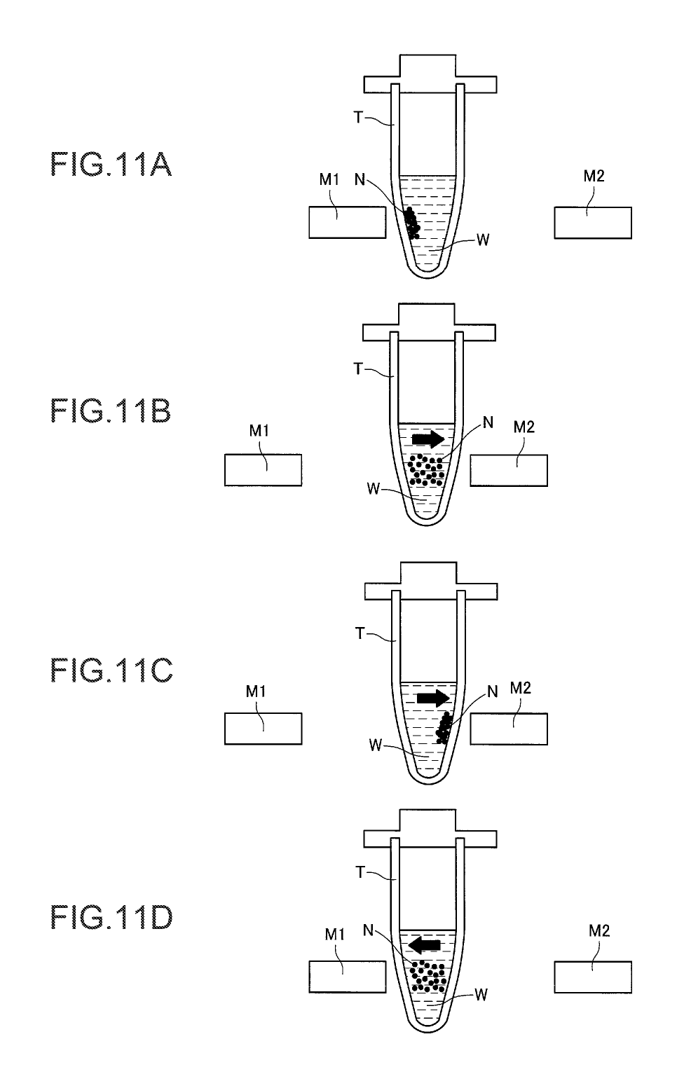

The followability of the nucleic acid-binding solid-phase carriers of Example 1 and Comparative Examples 1 and 2 with respect to the external magnetic field (with respect to the direction of application of the external magnetic field) was evaluated. Specifically, as shown in FIGS. 11A to 11D, a tube T to hold water W is prepared, and the nucleic acid-binding solid-phase carrier N was introduced into the water W. Subsequently, the tube T was placed between a magnet M1 and a magnet M2, and the magnets M1 and M2 were vibrated. Then, the frequency of vibration of the magnets M1 and M2 was changed within the range of 1 Hz to 7 Hz, and the followability of the nucleic acid-binding solid-phase carrier N with respect to the magnetic force was visually evaluated. As the tube T, a container having an inner diameter (maximum inner diameter) of 8 mm and made of polypropylene was used. As the magnets M1 and M2, permanent magnets were used. The average particle diameters of Example 1, Comparative Example 1, and Comparative Example 2 used in this experiment were 1.8 μm, 3.8 μm, and 1.0 μm, respectively.

FIGS. 11A to 11D are schematic views for illustrating an example in which the nucleic acid-binding solid-phase carrier N can follow the external magnetic field. In this example, as shown in FIGS. 11A to 11D, when the state of the magnets M1 and M2 is changed from a state where the magnet M1 comes close to the tube T (see FIG. 11A) to a state where the magnet M2 comes close to the tube T, all of the nucleic acid-binding solid-phase carriers N are separated from the inner wall of the tube T on the magnet M1 side (see FIG. 11B) and move to the inner wall of the tube T on the magnet M2 side (see FIG. 11C). Then, when the magnet M1 comes close to the tube T again, all of the nucleic acid-binding solid-phase carriers N are separated from the inner wall of the tube T on the magnet M2 side (see FIG. 11D). In this manner, all of the nucleic acid-binding solid-phase carriers N follow the external magnetic field.

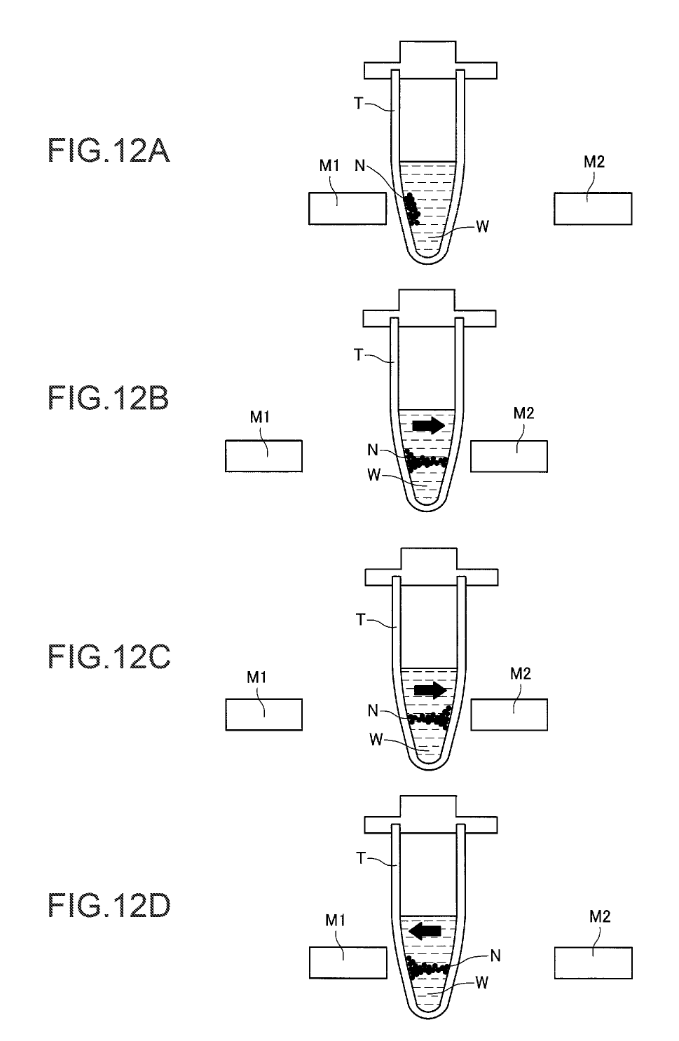

FIGS. 12A to 12D are schematic views for illustrating an example in which the nucleic acid-binding solid-phase carrier N cannot follow the external magnetic field. In this example, even if the state of the magnets M1 and M2 is changed from a state where the magnet M1 comes close to the tube T (see FIG. 12A) to a state where the magnet M2 comes close to the tube T, some of the nucleic acid-binding solid-phase carriers N remain on the inner wall of the tube T on the magnet M1 side (see FIGS. 12B and 12C). Then, before all of the nucleic acid-binding solid-phase carriers N move to the inner wall of the tube T on the magnet M2 side, the magnet M1 comes close to the tube T again (see FIG. 12D). Therefore, some of the nucleic acid-binding solid-phase carriers N cannot move to the inner wall of the tube T on the magnet M2 side. In this manner, some or all of the nucleic acid-binding solid-phase carriers N cannot follow the external magnetic field.

Table 1 is a table showing the followability of the nucleic acid-binding solid-phase carrier with respect to the external magnetic field. In Table 1, the case where all of the nucleic acid-binding solid-phase carriers N followed the magnetic force as shown in FIGS. 11A to 11D is indicated by “A”, and the case where some or all of the nucleic acid-binding solid-phase carriers N could not follow the magnetic force as shown in FIGS. 12A to 12D is indicated by “B”.

TABLE 1

| | Average particle | Frequency [Hz] | | | | | | |
|---|---|---|---|---|---|---|---|---|
| | diameter [μm] | 1 | 2 | 3 | 4 | 5 | 6 | 7 |
| Example 1 | 1.8 | A | A | A | A | A | A | A |
| Comparative | 3.8 | A | A | B | B | B | B | B |
| Example 1 | | | | | | | | |
| Comparative | 1.0 | B | B | B | B | B | B | B |
| Example 2 | | | | | | | | |

As shown in Table 1, the nucleic acid-binding solid-phase carrier of Example 1 followed the magnetic force when the frequency of vibration of the magnets M1 and M2 was from 1 Hz to 7 Hz. On the other hand, the nucleic acid-binding solid-phase carrier of Comparative Example 1 did not follow the magnetic force when the frequency of vibration of the magnets M1 and M2 was increased, and the nucleic acid-binding solid-phase carrier of Comparative Example 2 did not follow the magnetic force at all the tested frequencies. Therefore, it was found that the amorphous alloy powder containing Fe, Cr, Si, and B has higher followability with respect to the external magnetic field than the metal powder composed of iron oxide. In this experiment, the frequency of vibration of the magnets M1 and M2 was increased only up to 7 Hz, however, it is considered that the nucleic acid-binding solid-phase carrier of Example 1 can follow the magnetic force even when the frequency is higher than 7 Hz.

In Example 1, as the magnetic particle, an amorphous alloy powder containing Fe, Cr, Si, and B is used, and therefore, the coercivity is low. Due to this, as shown in FIGS. 11A to 11D, it is considered that the nucleic acid-binding solid-phase carrier is hardly transformed into a block when it moves from the magnet M1 side to the magnet M2 side and has high dispersibility. On the other hand, in Comparative Examples 1 and 2, as the magnetic particle, a metal powder composed of iron oxide is used, and therefore, the coercivity is high. Due to this, as shown in FIGS. 12A to 12D, it is considered that the nucleic acid-binding solid-phase carrier is easily transformed into a block when it moves from the magnet M1 side to the magnet M2 side.

### 5.4. Evaluation of Dependency of Ct Value on Average Particle Diameter

A nucleic acid extraction reaction (pre-treatment) was performed using the nucleic acid-binding solid-phase carrier of Example 1 having an average particle diameter of 1.8 μm, 3.2 μm, or 11 μm. Then, a real-time PCR reaction was performed by adding the resulting extraction liquid to a PCR reaction mixture, and a Ct value was obtained. The PCR was performed under the same conditions for the nucleic acid-binding solid-phase carrier having each average particle diameter using a DNA of an E. coli as a target nucleic acid.

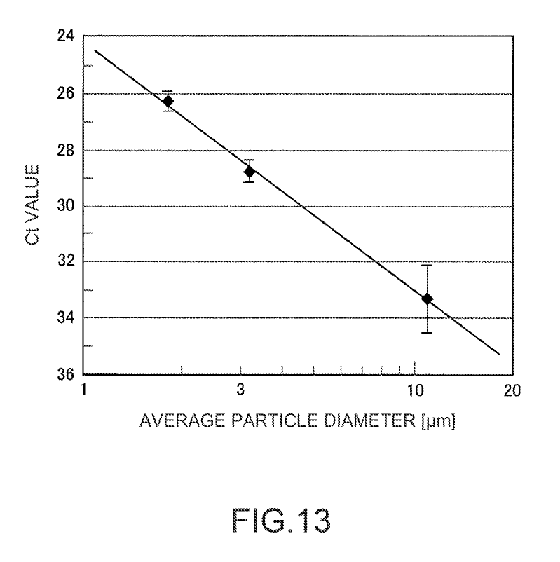

FIG. 13 is a graph showing a relationship between the average particle diameter of the nucleic acid-binding solid-phase carrier and the Ct value. As shown in FIG. 13, it was found that as the average particle diameter of the nucleic acid-binding solid-phase carrier is smaller, the Ct value is lower, and thus, the sensitivity of PCR can be enhanced. Incidentally, in FIG. 13, a variation in the particle diameter of the nucleic acid-binding solid-phase carrier is also shown.

Here, when the diameter of one nucleic acid-binding solid-phase carrier is represented by R, the total area A of the surface of the nucleic acid-binding solid-phase carrier included in the volume V is represented by the following formula (3).

A=4πR²*V/(4/3πR³)=3V/R (3)

According to the formula (3), when the particle diameter is changed from R₁ to R₂ (R₂<R₁), the total area A becomes (R₁/R₂) times. Therefore, as the average particle diameter of the nucleic acid-binding solid-phase carrier is decreased, the total area of the surface of the nucleic acid-binding solid-phase carrier is increased so as to increase the adsorption amount of the nucleic acid, and as a result, the Ct value is decreased as shown in FIG. 13.

The invention includes substantially the same configurations (for example, configurations having the same functions, methods, and results, or configurations having the same objects and effects) as the configurations described in the embodiments. Further, the invention includes configurations in which a part that is not essential in the configurations described in the embodiments is substituted. Further, the invention includes configurations having the same effects as in the configurations described in the embodiments, or configurations capable of achieving the same objects as in the configurations described in the embodiments. In addition, the invention includes configurations in which known techniques are added to the configurations described in the embodiments.

What is claimed is:

1. A nucleic acid-binding solid-phase carrier, comprising:
a magnetic particle of an amorphous metal containing Fe, Cr, Si, and B; and
a silicon oxide film provided on the surface of the magnetic particle.

2. The nucleic acid-binding solid-phase carrier according to claim 1, wherein the nucleic acid-binding solid-phase carrier has a saturation magnetization of 50 emu/g or more.

3. The nucleic acid-binding solid-phase carrier according to claim 1, wherein the nucleic acid-binding solid-phase carrier has a saturation magnetization of 100 emu/g or more and 150 emu/g or less.

4. The nucleic acid-binding solid-phase carrier according to claim 1, wherein the magnetic particle has an average particle diameter of 0.5 μm or more and 10 μm or less.

5. The nucleic acid-binding solid-phase carrier according to claim 1, wherein the silicon oxide film has an average thickness of 1 nm or more and 500 nm or less.

6. The nucleic acid-binding solid-phase carrier according to claim 1, wherein the magnetic particle is a soft magnetic body.

7. A nucleic acid extraction method, comprising:
adsorbing a nucleic acid on the nucleic acid-binding solid-phase carrier according to claim 1 in an adsorption liquid containing a chaotropic substance;
washing the nucleic acid-binding solid-phase carrier having the nucleic acid adsorbed thereon by placing the nucleic acid-binding solid-phase carrier having the nucleic acid adsorbed thereon in a washing liquid; and
eluting the nucleic acid in an eluent by placing the nucleic acid-binding solid-phase carrier having the nucleic acid adsorbed thereon in the eluent, wherein
the washing the nucleic acid-binding solid-phase carrier is performed by vibrating the nucleic acid-binding solid-phase carrier having the nucleic acid adsorbed thereon in the washing liquid by a magnetic force.

8. The nucleic acid extraction method according to claim 7, wherein the eluting the nucleic acid is performed by vibrating the nucleic acid-binding solid-phase carrier having the nucleic acid adsorbed thereon in the eluent by a magnetic force.
